(19) United States
(12) Patent Application Publication
(10) Pub. No.: US 2024/0360221 A1
(43) Pub. Date: Oct. 31, 2024

(54) ENGINEERING METHOD FOR MESENCHYMAL STEM CELLS BASED ON POLYVALENT ANTIBODY AND APPLICATION THEREOF

(71) Applicant: South China University of Technology, Guangzhou (CN)

(72) Inventors: Peng SHI, Guangzhou (CN); Tenghui YE, Guangzhou (CN); Xianghua ZHONG, Guangzhou (CN); Ran YAN, Guangzhou (CN); Xi LIU, Guangzhou (CN)

(21) Appl. No.: 18/664,845

(22) Filed: May 15, 2024

(51) Int. Cl. C07K 16/28; A61K 35/28; A61P 9/00; C12N 5/00; C12N 5/0775

(52) U.S. Cl. CPC C07K 16/2836; A61K 35/28; A61P 9/00; C12N 5/0006; C12N 5/0662; C07K 2317/35; C12N 2527/00

(57) ABSTRACT

An antibody monomer, including an antibody, and a first DNA monomer or a second DNA monomer, where the antibody is an anti-vascular cell adhesion molecule 1 (antiVCAM1) antibody. Nucleotide sequences of the first and second DNA monomers respectively consist of SEQ ID NO:1 and SEQ ID NO:2. A kit including the antibody monomer and an initiator including a nucleotide sequence consisting of SEQ ID NO: 3 is provided. This application also provides a polyvalent antibody including a first antibody monomer and a second antibody monomer, where the first antibody monomer includes the first DNA monomer and the antiVCAM1 antibody, and the second antibody monomer includes the second DNA monomer and the antiVCAM1 antibody. This application further provides a mesenchymal stem cell modified with the antibody monomer or the polyvalent antibody, and an application thereof in the treatment of tissue damage.

### REFERENCE TO AN ELECTRONIC SEQUENCE LISTING

The contents of the electronic sequence listing (SequenceListing.xml; Size: 4,165 bytes; and Date of Creation: Jul. 16, 2024) is herein incorporated by reference in its entirety.

### TECHNICAL FIELD

This application relates to biotechnology, and more particularly to an engineering method for mesenchymal stem cells based on polyvalent antibody and an application thereof.

### BACKGROUND

Mesenchymal stem cells (MSCs), generally referring to a group of multipotent stromal cells with tissue repair functions and a certain differentiation potential that can differentiate into multiple types of cells, have been widely used in the field of regenerative medicine due to excellent tissue repair activity, anti-inflammatory activity, and immunomodulatory activity. MSCs are weakly immunogenic due to the low expression of their constitutive major histocompatibility complex class I (MHC I), and the absence of MHC II and co-stimulatory molecules such as CD80, CD86 and CD40. Transplanted MSCs show good therapeutic effectiveness. Firstly, these transplanted MSCs have multidirectional differentiation potential, and produce specific extracellular matrix molecules, which can be differentiated into chondrocytes, bone, and adipocytes under different inducing conditions. In addition, the transplanted MSCs can improve the microenvironment of the damaged tissues through the paracrine pathway. Therefore, the MSCs have attracted extensive attention in the treatment of serious diseases, such as cardiovascular diseases, degenerative diseases, and bone injuries.

More than 1,300 MSC clinical studies worldwide have been included in Clinical trials until August 2022. Currently, one of the main challenges for MSC therapy is the effective delivery of cells into damaged tissues. Local injection into tissues or infusion into proximal vessels are two potential delivery routes. Because arterial walls are thin and pulsatile, local injection is dangerous for vascular injuries. However, intravascular infusion is minimally invasive, allows for repeat administration, and avoids the problems associated with secondary vascular injury and calcification. Unfortunately, in clinical studies, it has been found that after being infused through the vasculature, the transplanted MSCs ultimately successfully reach the target tissue is usually less than 1%. This low targeting efficiency severely limits the clinical efficacy of MSCs. Studies have shown that when MSCs reach the blood vessels in the damaged tissue, they adhere to the activated endothelium through the interactions between cell surface ligands and the overexpressed receptors on vascular endothelial cells and then migrate across the endothelial layer and colonize the damaged tissue. However, the MSCs surface ligands from different sources exhibit heterogeneous marker expression, and MSCs lose the key adhesion-related cell surface ligands during culture expansion in vitro, resulting in low adhesion and colonization efficiency of MSCs. Therefore, how to improve the adhesion efficiency of MSCs in damaged tissues has become a critical issue in clinical research.

Cell surface contains various biomolecules, and their environment recognition ability is closely associated with the cell function. Extensive researches have been conducted on the cell surface modification, in which the living cells are functionalized with a wide variety of exogenous biomolecules to achieve new molecular recognition functions. Functionalized cells have been extensively applied to immunotherapy, tissue engineering and stem cell homing. In recent years, the modification of MSCs through non-genetic engineering techniques has received increasing attention. It has been demonstrated that surface modification of the MSCs with a bioactive molecule having high affinity to pathological markers through a non-genetic engineering technique can improve the adhesion efficiency of intravascularly-delivered MSCs to target tissues and facilitate the tissue repair. Further, enzymatic transformation, covalent bonding, and hydrophobic insertion have been employed in the surface modification of MSCs. Moreover, it is required to ensure that the surface modification will not significantly alter or disturb the original composition and function of the cells. Therefore, it is of significance to achieve the desired molecular recognition function with as few exogenous biomolecules as possible. However, the existing functionalized MSCs are predominated by single molecule-functionalized cells, that is, a single biomolecule is present at individual points across the cell surface. These methods have exhibited remarkable effects, but they still have some limitations. Cell adhesion is achieved through polyvalent interaction, by comparison, the modification of MSC membranes with the adhesion molecule monomer is based on monovalent interaction. Due to the poor affinity of the monovalent interaction, the adhesion capacity of the modified MSCs still remains to be further enhanced.

### SUMMARY

A first object of this application is to provide an antibody monomer.

A second object of this application is to provide a kit.

A third object of this application is to provide a polyvalent antibody.

A fourth object of this application is to provide an application of the antibody monomer, the kit, or the polyvalent antibody in the cell engineering.

A fifth object of this application is to provide an engineering cell.

A sixth object of this application is to provide a cell engineering method.

A seventh object of this application is to provide an application of the engineering cell or the cell engineering method in the preparation of a product.

Technical solutions of this application are described as follows.

In a first aspect, this application provides an antibody monomer, comprising: an antibody, and a first DNA monomer or a second DNA monomer; wherein a nucleotide sequence of the first DNA monomer consists of SEQ ID NO: 1; and a nucleotide sequence of the second DNA monomer consists of SEQ ID NO: 2.

In an embodiment, an NH₂—-(CH₂)₆— group is linked to 3′ end of the nucleotide sequence of the first DNA monomer.

In an embodiment, an NH₂—-(CH₂)₆— group is linked to 5′ end of the nucleotide sequence of the second DNA monomer.

In an embodiment, the antibody is a molecule/protein that specifically adheres to a target protein. Preferably, the antibody is an antibody that specifically binds to a target antigen. More preferably, the antibody is an anti-vascular cell adhesion molecule 1 (anti-VCAM-1) antibody.

In an embodiment, the antibody monomer is prepared through the following steps:
(1) ligation of DNA monomer with a crosslinker mixing the DNA monomer, the crosslinker and dimethyl sulfoxide (DMSO) followed by reaction to obtain a DNA monomer-crosslinker conjugate;
(2) ligation of an antibody and the crosslinker mixing the antibody and the crosslinker followed by reacted to obtain an antibody-crosslinker conjugate; and
(3) mixing the DNA monomer-crosslinker conjugate with the antibody-crosslinker conjugate followed by reaction in the presence of a catalyst to obtain the antibody monomer.

In an embodiment, the crosslinker used in the ligation of the DNA monomer and the crosslinker includes at least one of S-4FB linker, SM (PEG) 2 (Thermo Fisher Scientific, CAS: 22102) and NHS-PEG-DBCO ester (Kangnuofu Biological Technology Co., Ltd., CAS: 1427004-19-0).

In an embodiment, the ligation of the DNA monomer and the crosslinker is performed under shaking at 20-38° C. for 1-3 h, preferably at 24-27° C. for 1-2 h, and more preferably at 25° C. for 2 h.

In an embodiment, the NH₂—-(CH₂)₆— group of the DNA monomer is linked to 4-FB.

In an embodiment, the crosslinker in the ligation of the antibody and the crosslinker includes S-HyNic or NHS-PEG-Azide (Thermo Fisher Scientific, CAS: 26130).

In an embodiment, the antibody is desalted before mixing with the crosslinker.

In an embodiment, the crosslinker is dissolved in DMSO before mixing with the antibody.

In an embodiment, the linkage of the antibody with the crosslinker is performed under shaking at 20-38° C. for 1-3 h; preferably, at 24-27° C. for 1-2 h; more preferably, at 25° C. for 2 h.

In an embodiment, the reaction after mixing the DNA monomer-crosslinker and antibody-crosslinker is performed under shaking at 20-38° C. for 1-3 h; preferably, at 24-27° C. for 1-2 h; more preferably, at 25° C. for 2 h.

In a second aspect, this application provides a kit comprising the antibody monomer in the first aspect.

In an embodiment, the kit further comprises an initiator.

In an embodiment, the initiator comprises a nucleotide sequence consisting of SEQ ID NO: 3.

In an embodiment, a cholesterol moiety is linked to the 3′ end of the nucleotide sequence of the initiator.

In a third aspect, this application provides a polyvalent antibody comprising a first antibody monomer and a second antibody monomer. The first antibody monomer comprises a first DNA monomer and a first antibody. The second antibody monomer comprises a second DNA monomer and a second antibody. The nucleotide sequence of first DNA monomer consists of SEQ ID NO:1, and the nucleotide sequence of second DNA monomer consists of SEQ ID NO:2. An NH₂—-(CH₂)₆— group is linked to 3′ end of the nucleotide sequence of the first DNA monomer. An NH₂—-(CH₂)₆— group is linked to 5′ end of the nucleotide sequence of the second DNA monomer. The first antibody and the second antibody are each an anti-vascular cell adhesion molecule 1 (antiVCAM1) antibody. The first antibody monomer and the second antibody monomer are connected by a hybridization chain reaction.

In an embodiment, the polyvalent antibody is prepared through mixing the first antibody monomer and the second antibody monomer with the initiator followed by reacting to obtain the polyvalent antibody.

In an embodiment, the final concentration of the initiator is 0.05-2 μM, preferably 0.05-1 μM, and more preferably 0.08-1 μM.

In a fourth aspect, this application provides an application of the antibody monomer, the kit, or the polyvalent antibody in the engineering cells.

In an embodiment, the cells comprise stem cells, and further comprises mesenchymal stem cells.

In a fifth aspect, this application provides cells surface-modified with the antibody monomer or the polyvalent antibody.

In an embodiment, the cells comprise stem cells, and further comprises mesenchymal stem cells.

In a sixth aspect, this application provides a cell engineering method, comprising:
(a) mixing to be engineered cells with an initiator followed by a first reaction to obtain a reaction product; and
(b) mixing the reaction product with the above antibody monomer followed by a second reaction to obtain the engineered cells; and
wherein the to be engineered cells are a mesenchymal stem cells, and the nucleotide sequence of the initiator consists of SEQ ID NO: 3.

In an embodiment, a cholesterol moiety is linked to the 3′ end of the nucleotide sequence of the initiator.

In an embodiment, the final concentration of the initiator is 0.05-3 μM, preferably 0.05-2 μM, and more preferably 0.05-1 μM.

In an embodiment, the first reaction is performed under shaking at 20-37° C. for 15-30 min, preferably at 20-25° C. for 20-30 min, and more preferably at 25° C. for 20 min.

In an embodiment, the second reaction is performed under shaking at 20-37° C. for 2-4 h, preferably at 20-25° C. for 2-3 h, and more preferably at 25° C. for 3 h.

In an embodiment, the cells comprise stem cells, preferably mesenchymal stem cells.

In a seventh aspect, this application provides an application of the cells surface-modified with the antibody monomer or the polyvalent antibody or the engineered cells in the tissue repair and/or in the enhancement of the adhesion ability of cells to damaged tissue.

In an embodiment, the tissue comprises blood vessels.

The beneficial effects of this application are described as follows.

The antibody monomer provided herein is obtained by linking a DNA sequence to a protein, where the DNA sequence is specifically designed according to the programmability of DNA. By modifying the cell surface with the antibody monomer, the cell adhesion to the damaged tissue can be improved, thereby promoting the tissue repair.

The polyvalent antibody provided herein is formed from antibody monomers through the hybridization chain reaction in the presence of a DNA initiator under artificially-controllable conditions. The modification of the mesenchymal stem cells with the polyvalent antibody can greatly enhance the cell adhesion ability to blood vessels of the damaged tissue site.

In the engineered cell provided herein, the polyvalent antibody adheres to the cell surface to achieve the multivalent functionalization of the cell, which can greatly enhance the adhesion ability to blood vessels in the damaged tissue site through the high affinity of the polyvalent action without affecting the proliferation rate and the function of secreting cytokines.

The engineering method of MSCs proposed herein only needs to connect DNA monomers with antibodies in advance. Firstly, the DNA initiator is added to the cell suspension, and then the DNA initiator is connected to the surface of MSCs by hydrophobic intercalation. Secondly, the antibody monomers (DM1-antiVCAM1, DM2-anti VCAM1) are added, and the complementary DNA is self-assembled on the surface of MSCs via the DNA hybridization chain reaction to guide the formation of polyvalent antibodies. The whole engineering process can be completed in a few hours in a PBS system, which is simple and efficient, and has less effect on the cells. After the engineering modification, the high affinity provided by the multivalent interaction between the polyvalent antibody and vascular epithelial cells greatly enhances the adhesion of MSCs to vascular epithelial cells, thereby enhancing the therapeutic effectiveness of MSCs in the tissue repair. The construction of multimer molecules on the cell surface provides a new idea to enhance the targeted adhesion ability of MSCs.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 schematically shows secondary structures of DNA initiator (DI), DNA monomer 1 (DM1) and DNA monomer 2 (DM2);

FIG. 2 is a polyacrylamide gel electrophoresis map of a DNA multimer produced in Example 1 of the present disclosure;

FIG. 3A is a polyacrylamide gel electrophoresis map of DM1-antiVCAM1 monomer and DM2-antiVCAM1 monomer, where “V” represents antiVCAM1;

FIG. 3B shows ultraviolet absorption spectra of DM1-antiVCAM1 monomer and DM2-antiVCAM1 monomer;

FIG. 4A shows flow cytometric analysis results of C166 cells respectively stained with fluorescein 5-isothiocyanate (FITC)-conjugated antiVCAM1 and FITC-conjugated DNA-antiVCAM1;

FIG. 4B shows flow cytometric analysis results of K562 cells respectively stained with FITC-conjugated antiVCAM1 and FITC-conjugated DNA-antiVCAM1;

FIG. 4C shows FITC fluorescence intensity of the stained C166 cells and K562 cells;

FIG. 5 is a polyacrylamide gel electrophoresis map of polyvalent antibodies prepared in Example 4 of the present disclosure;

FIG. 6 schematically shows an engineering process of MSCs according to an embodiment of the present disclosure;

FIG. 7 is a laser scanning confocal microscopy (LSCM) image of engineered MSCs according to an embodiment of the present disclosure;

FIG. 8A shows flow cytometric analysis results of the engineered MSCs;

FIG. 8B shows FITC fluorescence intensity of the engineered MSCs, where “****” represents p<0.001;

FIG. 9 is a schematic diagram of a device used in the rolling adhesion experiment;

FIGS. 10A-10B show statistical results of the number of MSCs adhering to C166 cells under different shear stress conditions; where 10A: the number of MSCs; and 10B: enhancement efficiency;

FIGS. 11A-11B show the number of MSCs adhering to C166 cells in the presence of different numbers of modified DIs; where 11A: the number of MSCs; and 11B: enhancement efficiency;

FIGS. 12A-12C are fluorescence microscope images of MSCs adhering to the C166 cells, where 12A is a control group, 12B is a monomer group, and 12C is a multimer group;

FIG. 13 schematically depicts a device used in the adhesion and migration experiment in Example 10 of the present disclosure;

FIGS. 14A-14C are three-dimensional confocal microscopy images of C166 cells in a flow chamber, where 14A: the control group at 0 and 24 h; 14B: the monomer group at 0 and 24 h; and 14C: the multimer group at 0 and 24 h;

FIGS. 15A-15C show quantification results of MSCs migrating into collagen I under C166 cells; where 15A is a confocal microscopy image, 15B shows statistical counting results of MSCs, and 15C shows statistical results of enhancement efficiency;

FIG. 16 shows statistical results of cell counting kit-8 (CCK-8) assay of the engineered MSCs;

FIG. 17 shows paracrine function test results of the engineered MSCs;

FIGS. 18A-18C show in vivo imaging results of mice with acute ear inflammation after injected with the engineered MSCs, where 18A is the control group, 18B is the monomer group, and 18C is the multimer group;

FIGS. 19A-19C are confocal fluorescence microscopy images of mouse ears, where 19A is the control group, 19B is the monomer group, and 19C is the multimer group;

FIGS. 20A-20B show statistical results of the number of the engineered MSCs in the ears of mice with inflammation; where 20A shows statistical results of the number of MSCs, and 20B shows statistical results of enhancement efficiency; and

FIGS. 21A-21C are fluorescence images of frozen sections of ears of the mice with inflammation, where 21A is the control group, 21B is the monomer group, and 21C is the multimer group.

### DETAILED DESCRIPTION OF EMBODIMENTS

The present disclosure will be further described in detail in conjunction with embodiments.

These embodiments are used only to illustrate the present disclosure, and are not intended to limit the disclosure.

The materials and reagents used in these embodiments are commercially available unless otherwise specified.

Example 1 Verifying that DNA multimer can be synthesized from DM1 and DM2 via a hybridization chain reaction (HCR) in the presence of DI

Example 1 was set for confirm the preparation of DNA multimer through the hybridization chain reaction of DNA Monomer 1 (DM1) and DNA Monomer 2 (DM2) in a solution phase under the initiation action of the DNA initiator (DI).

Base sequence of DM1 was 5′-GGTTTAGGTAGGAGTGGGATGAGGCCAAATCCTCATCCCACTCCTACC-3′(SEQ ID NO: 1), with a NH₂—-(CH₂)₆— group attached to the 3′ end.

Base sequence of DM2 was 5′-CCTCATCCCACTCCTACCTAAACCGGTAGGAGTGGGATGAGGATTTGG-3′(SEQ ID NO: 2), with a molecular group NH₂—-(CH₂)₆— attached to the 5′ end.

Nucleotide sequence of DI was 5′-CCTCATCCCACTCCTACCTAAACCTTTTTTTTTTTTTTTTTTTT-3′ (SEQ ID NO: 3), with a cholesterol group attached to the 3′ end. DM1, DM2 and DI were synthesized by Sangon Biotech Co., Ltd (Shanghai, China).

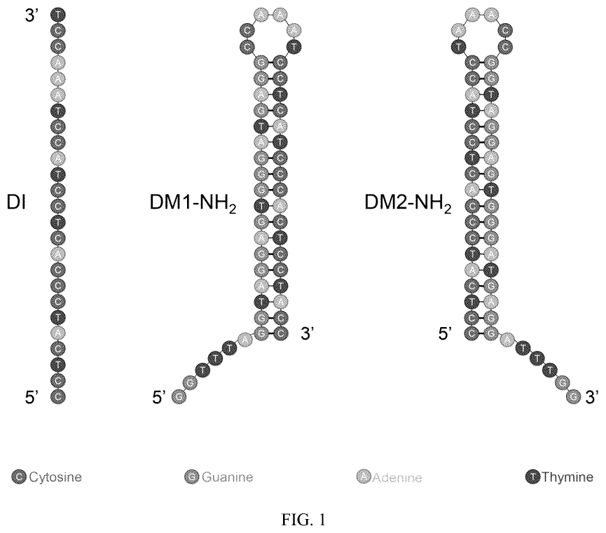

DM1 and DM2 each had a hairpin structure as shown in FIG. 1.

A preparation method for the DNA multimer included the following steps.
(1) DM1, DM2, and DI were dissolved in 1× phosphate-buffered saline (PBS) (pH 7.4), respectively, to obtain a 10 μM DM1 solution, a 10 μM DM2 solution, and a 10 μM DI solution.
(2) The DM1 solution and the DM2 solution were separately annealed at 95° C. for 10 min.
(3) 5 μL of the annealed DM1 solution, 5 μL of the annealed DM2 solution and 0.6 μL of the DI solution were mixed well, and reacted under shaking at 25° C. for 3 h to yield the DNA multimer.

Whether the DNA multimer had been successfully synthesized was verified by polyacrylamide gel electrophoresis, where a 8% separating gel was used, and the electrophoresis was performed at a voltage of 90 v for 60 min. A control group was set, in which the annealed DM1 solution and the annealed DM2 solution were included, and the DI solution was absent.

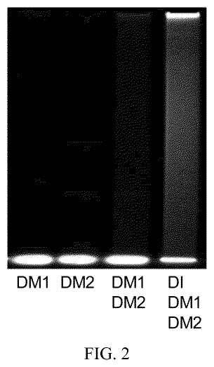

The results were shown in FIG. 2. Only in the presence of DI solution, DM1 and DM2 could undergo the hybridization chain reaction to form the DNA multimer in the solution. The control group without the DI was almost unable to yield the DNA multimer.

### Exampe 2 DNA-Antibody Conjugation

DM1-antiVCAM1 monomers and DM2-antiVCAM1 monomers were prepared by ligating DNA (DM1 or DM2) to antiVCAM1.

The prparation method of DM1-antiVCAM1 monomers included the following steps.

(1) Ligation of DM1 with S-4FB Crosslinker

S-4FB Crosslinker (4-FB, SoluLink* bioconjugation technology, catalog: S-1004-010) was bound to the NH₂—-(CH₂)₆— group at the 3′ end of DM1. Firstly, 15 OD of DM1 was dissolved in 60 μL of buffer M (1×PBS, pH 8.0) to obtain DM1 solution. 1 mg of 4-FB was dissolved in 40 μL of anhydrous dimethyl sulfoxide (DMSO) to obtain 4-FB solution. 30 μL of DMSO and 6.5 μL of the 4-FB solution were added to the DM1 solution, mixed well, and reacted with shaking at 25° C. for 2 h. At the end of the reaction, the reaction mixture was added to a 10 kDa ultrafiltration tube, following by adding the buffer C (1×PBS, pH 6.0) to make the total volume of 500 μL, placing in a centrifuge at 15,000× g for 10 min, and removing the waste solution. The ultrafiltration process was repeated for 2 times. Then, the ultrafiltration tube was inverted in a new collection tube, placed in a centrifuge at 1500× g for 2 min to collect the DM1-4FB, and quantified to 100 μL with the buffer C, thereby obtaining the high-purity DM1-4FB.

(2) Ligation of AntiVCAM1 with S-HyNic

100 ug of antiVCAM1 (Biolengend, catalog: 105706) was dissolved with 100 uL buffer M. The antibody was desalted using a desalting column with 7MWCO, according to the instruction manual of the desalting column. The desalted antibody was quantified to 100 μL using the buffer M to obtain antiVCAM1 solution. 100 ug of S-HyNic Crosslinker (S-HyNic, SoluLinkR bioconjugation technology, catalog: S-1002-105) was dissolved in 35 μL of DMSO to obtain the S-HyNic solution. 3 μL of the S-HyNic solution was added to in the antiVCAM1 solution, mixed well, and reacted with shaking at 25° C. for 2 h. After the reaction, the reaction mixture was added into a 30 kDa ultrafiltration tube, diluted with the buffer C to a volume of 500 μL, and centrifuged at 15,000× g for 10 min, and the bottom filtrate was discarded. The ultrafiltration process was repeated for 2 times. Then, the ultrafiltration tube was inverted in a new collection tube, placed in a centrifuge and centrifuged at 1,500× g for 2 min to collect the antiVCAM1 solution, and diluted to 100 μL with the buffer C, thereby obtaining the high-purity antiVCAM1-S-HyNic.

(3) Ligation of antiVCAM1 with DM1-4FB

10 μL of DM1-4FB obtained in step (1) was added into antiVCAM1-S-HyNic obtained in step (2), following by adding 12 μL of catalyst 10× Turbolink Catalyst Buffer (SoluLink bioconjugation technology, catalog: S-2006-105), mixing well, and reacting with shaking at 25° C. for 2 h. At the end of the reaction, the reaction mixture was added to a 50 kDa ultrafiltration tube, following by adding 1×PBS (pH 7.4) to make the total volume of 500 μL, centrifuging at 14,000× g for 10 min in a centrifuge, and removing the bottom filtrate. The ultrafiltration process was repeated for 5 times. Then, the ultrafiltration tube was inverted into a new collection tube, placed in a centrifuge at 1500× g for 2 min to collect DM1-antiVCAM1, and quantified to 100 μL with 1×PBS, thereby obtaining the high-purity DM1-antiVCAM1 monomer.

DM2-antiVCAM1 monomer was prepared in the same way as DM1-antiVCAM1 monomer, with the only difference being the replacement of DM1 with DM2.

The prepared DM1-antiVCAM1 monomer and DM2-antiVCAM1 monomer were characterized using polyacrylamide gel electrophoresis, with antiVCAM1 and antiVCAM1-S-HyNic obtained in step (2) as a control group for simultaneous characterization. The specific steps of polyacrylamide gel electrophoresis were as follows.

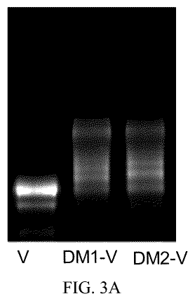

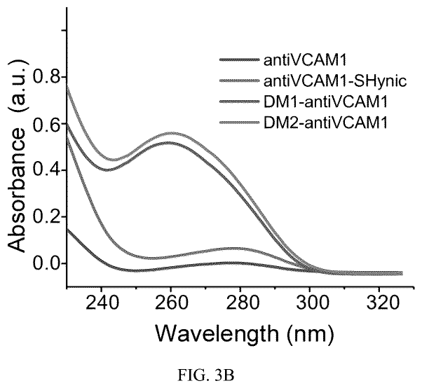

1 μL of antiVCAM1, DM1-antiVCAM1 monomer and DM2-antiVCAM1 monomer were each taken, respectively, following by adding 1 μL of 5x risperdal protein buffer (Band-Nonow Risperdal Protein Sampling Buffer (Sangon Biotech Co., Ltd (Shanghai, China), C506058), respectively, adding 3μL 1×PBS (pH 7.4), mixing well, and incubating at 95° C. for 10 min to denature the antibody. The SDS-PAGE electrophoresis gel and 8% separator gel were prepared. The samples were uploaded to start electrophoresis under electrophoresis conditions of voltage 100V for 60 min, and imaged. The electrophoretic gel images showed the electrophoretic bands of DM1-antiVCAM1 monomer and DM2-antiVCAM1 monomer were higher than those of antiVCAM1 as shown in FIG. 3A, i.e., the molecular weights of DM1-antiVCAM1 monomer and DM2-antiVCAM1 monomer were higher than those of antiVCAM1, indicating that the antiVCAM1 ligated to the DNA.

Absorbance values of antiVCAM1, antiVCAM1-S-HyNic, DM1-antiVCAM1 monomer and DM2-antiVCAM1 monomer at 260-280 nm were detected by an ultra-micro spectrophotometer, respectively. FIG. 3B showed that DNA had an absorbance peak at 260 nm, protein had an absorbance peak at 280 nm, and DM1-antiVCAM1 monomer and DM2-antiVCAM1 monomer had absorbance peaks at 260 nm and 280 nm, which further indicated that the attachment of antiVCAM1 to DNA was successful.

### Example 3 Verifying Specificity and Non-Specificity of AntiVCAM1 After Ligation of DNA

This Example was set for testing whether specificity and non-specificity of anti VCAM1 were altered after that antiVCAM1 was ligated to DNA.

The specific experimental procedure was as follows.

Endothelial cell line C166 cells (overexpressing VCAM 1) and control cell line K562 cells (not expressing VCAM 1) were taken, and each divided into three groups to form six groups (grouped as Table 1). Each group had 1×10⁶ cells and had three replicates. The well-grown cells were centrifuged at 1000 rpm for 5 min following by removing supernatant and washing twice with 1×PBS (pH 7.4) to remove the residual medium. The cells were resuspended with 200 μL of 1×PBS, and the corresponding antibody was added according to Table 1. The antibody-cell mixture was mixed homogeneously, and then incubated on ice for 20 min. At the end of the reaction, the cell suspension was centrifuged at 1000 rpm for 5 min, and the resultant supernatant was discarded, and the cells were washed twice with 1×PBS to remove the unreacted antibody. FITC fluorescence intensity of each group of cells was detected by flow cytometer.

TABLE 1
Experimental grouping

| Group name | Antibody | Dosage |
|---|---|---|
| Control group | — | — |
| (C166) | | |
| Experimental | antiVCAM1 | 2 μL |
| group (C166) | | |
| Experimental | DNA-antiVCAM1 (DM1-antiVCAM1 | 2 μL |
| group (C166) | monomer or DM2-antiVCAM1 monomer | |
| | obtained in Example 2) | |
| Control group | — | — |
| (K562) | | |
| Experimental | antiVCAM1 | 2 μL |
| group (K562) | | |
| Experimental | DNA-antiVCAM1 (DM1-antiVCAM1 | 2 μL |
| group (K562) | monomer or DM2-antiVCAM1 monomer | |
| | obtained in Example 2) | |

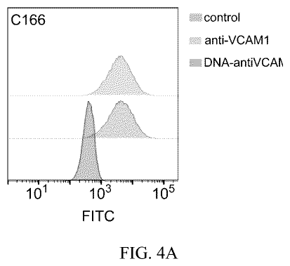

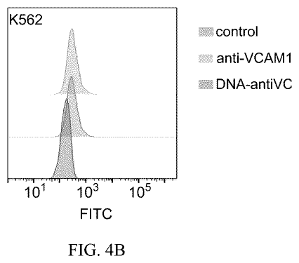

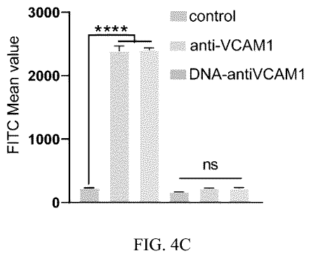

FIGS. 4A-4C showed that staining of antiVCAM1 and DNA-antiVCAM1 binding on C166 cells overexpressing VCAMI were consistent, and the fluorescence intensity was consistent, indicating that DNA ligation did not alter the specificity of anti VCAM1. Staining results of antiVCAM1 and DNA-antiVCAM1 binding on K562 cells not expressing VCAMI were consistent, and the fluorescence intensity was consistent, indicating that DNA ligation did not alter non-specificity of antiVCAM1.

### Example 4 Synthesis of Polyvalent Antibodies from HCR Reaction of DM1-AntiVCAM1 Monomer, DM2-AntiVCAM1 Monomer and DI

This Example was set for preparing an adherent polyvalent antibody by DM1-anti VCAM1 monomer and DM2-antiVCAM1 monomer with DI in the presence of PBS solution.

The preparation process was as follows.
(1) DM1-antiVCAM1 monomer (prepared in Example 2), DM2-antiVCAM1 monomer (prepared in Example 2), and DI were taken and dissolved in 1×PBS (pH 7.4) to obtain DM1-antiVCAM1 monomer solution, DM2-antiVCAM1 monomer solution, and DI solution with a concentration of 10 μM, respectively.

1(2) 3 μL of the DM1-antiVCAM1 monomer solution, DM2-antiVCAM1 monomer solution, and 0.6 μL of DI solution in step (1) were mixed, and reacted under shaking at 25° C. for 3 h to obtain the adhesion polyvalent antibody.

3 μL of the DM1-antiVCAM1 monomer solution and DM2-antiVCAM1 monomer solution from step (1) were taken respectively, mixed well, and reacted under shaking at 25° C. for 3 h, thereby obtaining the mixture used as control group 1. 3 μL of DM1-antiVCAM1 monomer solution in step (1) was taken and reacted under shaking at 25° C. for 3 h, thereby obtaining the mixture used as control group 2. 3 μL of DM2-anti VCAM1 monomer solution from step (1) was reacted under shaking at 25° C. for 3 h, thereby obtaining the mixture used as control group 3.

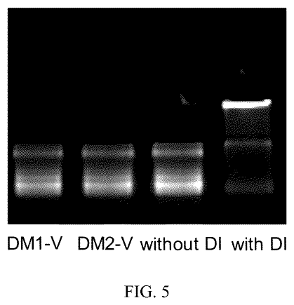

Polyacrylamide gel electrophoresis was used to detect the prepared adhesion molecule multimer and the mixtures prepared in control groups 1-3 to verify whether the adhesion molecule multimer were formed. The results were shown in FIG. 5. When there was no DI present in the reaction system, no hybridization chain reaction occurs to form polyvalent antibodies. Only in the presence of DI, the molecular weights of the reaction products became significantly larger, indicating that DM1-antiVCAM1 and DM2-antiVCAM1 could undergo hybridization chain reaction to form polyvalent antibodies.

### Example 5

1. Assembly of DM1-antiVCAM1 monomer and DM2-antiVCAM1 monomer on MSCs, i.e. self-assembly of antiVCAM1 multimer on MSCs

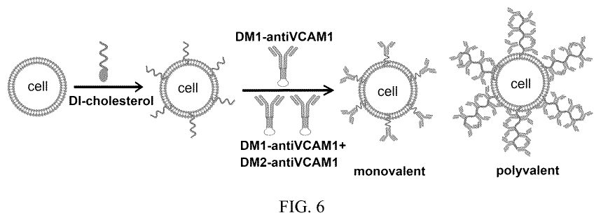

An engineering method 1 for MSCs based on polyvalent antibodies included the following steps.
(1) MSCs from mouse bone marrow (Saibaikang (Shanghai) Biotechnology Co., Ltd, catalog: MIC-iCell-s018) (all the generations of MSCs in the disclosure were 4˜6 generations) was digested from a petri dish, resuspended with 1×PBS (pH 7.4), and centrifuged at 1000 rpm for 5 min, following by removing the supernatant. This above treatment process repeated 1 time. The residual medium in the MSC suspension was washed away.
(2) 1×10⁶ MSCs was counted and resuspended in 200 μL of 1×PBS, following by adding DI (final concentration of 0.5 μM), mixing well, and reacting under shaking at 25° C. for 20 min. After 10 min of reaction, the MSCs solution was blown using the pipette gun to mix well, in order to achieve a better reaction effect.
(3) After the reaction, the MSCs solution was centrifuged at 1000 rpm for 5 min following by removing the supernatant, adding 1 mL of 1×PBS to resuspend, centrifuging at 1000 rpm for 5 min, and removing the supernatant. This above treatment process repeated 1 time to wash away the unreacted DI.
(4) The MSCs obtained in step (3) was resuspended with 200 μL of 1×PBS following by adding 3 μL of DM1-antiVCAM1 monomer and 3 μL of DM2-anti VCAM1 monomer (prepared in Example 2), blowing and mixing uniformly with a pipette gun every 1 h, and reacting under shaking at 25° C. for 3 h.
(5) After the reaction, the reaction solution was centrifuged at 1000 rpm for 5 min following by removing the supernatant, adding 1mL 1×PBS to resuspend, centrifuging at 1000 rpm for 5 min, and removing the supernatant. This above treatment process repeated 1 time to wash away the unreacted DNA-antibody, thereby obtaining the engineered MSCs as shown in FIG. 6.
2. Assembly of only DM1-antiVCAM1 monomers on MSCs

The engineering method 2 of MSCs was almost the same as the engineering method 1. The engineering method 2 differs from the engineering method 1 only in that only 3 μL of DM1-antiVCAM1 monomer was added in step (4).
3. Assembly of only DM2-antiVCAM1 monomer on MSCs

The engineering method 3 of MSCs was almost the same as the engineering method 1. The engineering method 3 differs from the engineering method 1 only in that only 3 μL of DM2-antiVCAM1 monomer was added in step (4).
4. Self-assembly of DM2-antiVCAM1 monomer and DM1-4FB on MSCs

The engineering method 4 of MSCs was almost the same as the engineering method 1. The engineering method 4 differs from the engineering method 1 only in that that 3 μL of DM2-antiVCAM1 monomer and 3 μL of DM1-4FB (obtained in step (1) of Example 2) were added in step (4).

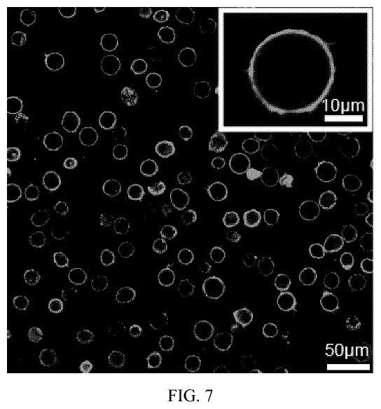

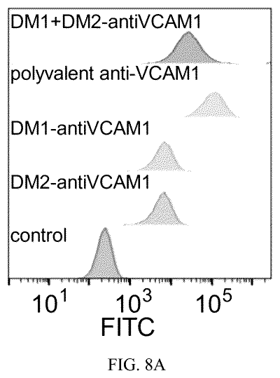

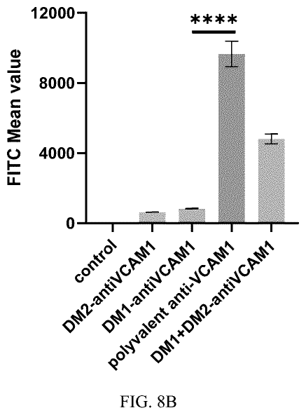

The MSCs engineered with polyvalent antibody in the engineering method 1 were resuspended with 1×PBS and imaged under a confocal microscope to characterize the MSCs, which showed that the polyvalent antibodies were modified with modifications on the MSC cell membrane (FIG. 7). The MSCs obtained by the above engineering methods 1-4 were further characterized by the flow cytometer. The FITC fluorescence intensity of the engineered MSCs was determined as shown in FIGS. 8A-8B, where the fluorescence intensity of MSCs engineered by polyvalent antibody was about 8 times higher than that of MSCs engineered by only antibody monomer.

### Example 6 Quantification of DI Modified on MSCs

This Example was arranged for quantifying the average number of DI inserted into each MSC after reaction of different concentrations of DI with the MSC.

The specific experimental process was as follows.
(1) Preparation of DI-CS-FAM (complementary chain of DI, which could be bound to DI with a FAM fluorescent group synthesized by Sangon Biotech Co., Ltd (Shanghai, China)) standards. 500 μL of DI-CS-FAM solution at concentrations of 10nM, 20 nM, 50 μM, 100 nM, 200 nM, and 500 nM, respectively, were prepared using RIPA lysis buffer (Sangon Biotech Co., Ltd (Shanghai, China), catalog: C500005)
(2) Well-grown mouse bone marrow MSCs were bought and divided into 8 groups, which included 7 experimental groups and 1 control group with three replicates in each group. Each group containing 1×10⁶ cells. The MSCs were centrifuged at 1000 rpm for 5 min, and the supernatant was removed. The cells were washed twice with PBS to wash away the residual culture medium.
(3) The cells of each group were resuspended with 200 μL of 1×PBS following by adding DI solution to make the final concentration of 0.05 μM, 0.075 μM, 0.1 μM, 0.25 μM, 0.5 μM, 1 μM, 2 μM (corresponding to the experimental groups 1-7), and 0 μM (the control group), mixing well, and reacting under shaking at room temperature (25° C.) for 20 min. At 10 min, the cells were blown once.
(4) At the end of the reaction, the cells were centrifuged at 1000 rpm for 5 min, the supernatant was removed, and the cells was washed twice with 1×PBS to remove the unreacted DI.
(5) The cells of each group were resuspended with 200 ul of PBS following by adding DI-CS-FAM solution to make the final concentration of 0.05 μM, 0.075 μM, 0.1 μM, 0.25 μM, 0.5 μM, 1 μM, 2 μM (corresponding to the experimental groups 1-7) and 0 μM (the control group), mixing well and reacting under shaking at room temperature for 20 min. At 10 min, the cells were blown once.
(6) At the end of the reaction, the cells were centrifuged at 1000 rpm for 5 min, the supernatant was removed, and the cells was washed twice with 1×PBS to remove the unreacted DI-CS-FAM.
(7) Cell counting was performed for each group of cells. Then the cells of each group were resuspended by 500 μL of RIPA lysis buffer and mixed well, and the cells were lysed sufficiently.
(8) Fluorescence intensity of FAM in the DI-CS-FAM standard and the lysed cell suspension was quantified by a fluorescence spectrophotometer.
(9) Quantitation of DI on the surface of each cell

According to the fluorescence intensity of the standards, the standard curve was obtained. The fluorescence intensity of cell suspensions in each group was substituted into the equation of the standard curve to determine the concentration of DI-CS-FAM of cell suspensions in each group, which was the concentration of DI inserted into the cells. Then, the concentration was converted into the number of DI according to the Avogadro's constant, and then the number of DI was divided by the number of cells in each group, thereby obtaining the number of DI inserted on each cell in each group.

The DI number on each cell in each group was finally calculated as shown in Table 2. The average number of DI inserted on MSCs gradually increased as the concentration of DI solution increased.

TABLE 2
Average number of DI on MSCs in each group

| | | |
|---|---|---|
| Group name | Concentration of DI | DI number on each cell |

| | | | |
|---|---|---|---|
| Experimental | 0.05 | μM | 1.45 × 10⁶ |
| group 1 | | | |
| Experimental | 0.075 | μM | 2.03 × 10⁶ |
| group 2 | | | |
| Experimental | 0.1 | μM | 3.3 × 10⁶ |
| group 3 | | | |
| Experimental | 0.25 | μM | 6.2 × 10⁶ |
| group 4 | | | |
| Experimental | 0.5 | μM | 1.31 × 10⁷ |
| group 5 | | | |
| Experimental | 1 | μM | 3.33 × 10⁷ |
| group 6 | | | |
| Experimental | 2 | μM | 4.37 × 10⁷ |
| group 7 | | | |
| Control group | 0 | μM | 0 |

### Example 7 Adhesion Assay of Engineered MSCs to Vascular Endothelial Cells

This Example simulated the shear stress associated with the blood flow in the vasculature in vivo and designed a flow adhesion assay to observe the adhesion of polyvalent antibody-engineered MSCs in Example 5 to vascular endothelial cells.

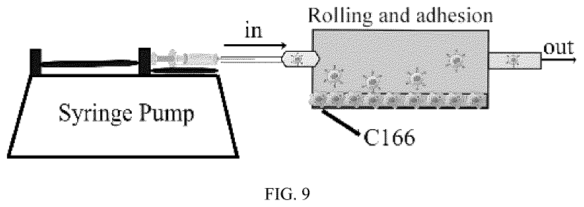

A schematic diagram of the assay device was shown in FIG. 9. The principle of the device was as follows. A precision syringe pump was used to provide a stable shear stress. Vascular endothelial cells C166 overexpressing VCAM1 were seeded in a flow chamber. The engineered or unmodified MSCs were resuspended in 1×PBS and loaded in the syringe. The syringe pump pushed cells in the syringe into the flow chamber from the inlet thereof, rolling and adhering in the flow chamber seeded with C166 cells. Unadhered MSCs flowed out from the outlet of the flow chamber. After the flow adhesion experiment, fluorescence microscopy was used to observe and quantify the MSCs adhered to C166 cells.

The detailed experimental procedures were as follows.
(1) C166 cells were seeded in the flow chamber. C166 cells were stained with DID using the staining kit (Beyotime Biotechnology Co., Ltd. (Shanghai, China)). The staining process was as follows. The cells to be stained were taken out of the petri dish. The cells were centrifuged at 1000 rpm for 5 min, the supernatant was removed, and the cells were washed twice with PBS to remove the residual medium. According to the instructions, staining solution was prepared. 1mL of the staining solution could dye 2×10⁶ cells. The cells to be stained were resuspended with the staining solution, mixed well, and incubated at 37° C. for 15 min. After incubation, the cells were centrifuged at 1000 rpm for 5 min, the supernatant was removed, and the cells were washed twice with PBS to remove residual staining solution. After staining, 2.5×10⁵ cells were resuspended in 100μL DMEM complete medium, added to the flow chamber by a pipette gun, and left at 37° C., 5% CO₂ environment for 4 h. The cells were observed to be adhered to the wall under the microscope, and 60mL of fresh DMEM complete medium was added on both sides of the wells of the flow chamber. The cells were continuously incubated for 10 h until the cells reached more than 90% coverage, and at this time, the C166 cells were used for rolling and adhesion assays.
(2) 1×10⁶ well-grown MSCs were stained with DIO using the staining kit (Beyotime) in the flow chamber. The staining method was the same as step (1). After the staining was finished, the cells were resuspended in 200 μL of 1×PBS, added with DI (the final concentration of 0.6 μM), mixed well, and oscillated at 25° C. for 20 min. At 10 min of reaction, the reaction mixture was blown out to achieve a better reaction effect. After the reaction, the cells were centrifuged at 1000 rpm for 5 min, the supernatant was removed, and the cells were resuspended in 1 mL of 1×PBS, centrifuged at 1000 rpm for 5 min, the supernatant was removed. Then, the treatment process was repeated to wash away the unreacted DI. The DI number inserted on the surface of the MSCs was determined to be 1.6×10⁷.
(3) MSC surface-modified polyvalent antibody

MSCs were suspended in 200 μL of PBS and added with 3 μL of DM1-anti VCAM1 and DM2-antiVCAM1 (prepared in Example 2), respectively. MSCs were mixed well with a pipette gun, reacted with oscillating at 25° C. for 3 h, and mixed well by blowing up every 1 h. At the end of the reaction, the MSCs were centrifuged at 1000 rpm for 5 min, and the supernatant was removed, the MSCs were suspended in 1 mL of 1×PBS, centrifuged at 1000 rpm for 5 min, and the supernatant was removed. The treatment was repeated once to wash away the unreacted antibody to obtain the polyvalent antibody-engineered MSCs (multimer group). The engineered MSCs obtained by only adding 3 μL of DM1-antiVCAM1 were used as the monomer control group (monomer group, i.e., MSC surface was engineered with antibody monomer). MSCs without any modification were used as the control group.
(4) The MSCs in the multimer group, monomer group, and control group were resuspended in 10 mL of 1×PBS and loaded in a 10 mL syringe to perform rolling adhesion assays using the device shown in FIG. 9.
(5) The device was connected according to the device schematic diagram. The driving force of the precision syringe pump was adjusted to provide different flow rates, thereby obtaining different shear stresses. The relationship between shear stress and flow rate was calculated according to the following formula: Σ=η·131.6·Φ, where τ is the shear stress, η is the kinetic viscosity, and Φ is the flow rate. The three shear stress conditions of 2 dyn/cm², 4 dyn/cm² and 8 dyn/cm² were set. Three replicate assays were performed for each shear stress.
(6) When the flow of 10 mL of cell suspension was finished, the remaining cell suspension in the chamber was removed, fluorescence imaging was immediately performed by a fluorescence microscope to observe the MSCs adhering to the C166 cells. 5-7 pictures were taken. Image J was used to count the number of MSCs adhering to the C166 cells and calculate the enhancement efficiency. The enhancement efficiency was calculated using the following formula: enhancement efficiency (%)=(monomer group value or multimer group value−control group value)/control group value×100%. The differences in the number of cells adhering to C166 cells between the antibody monomer-engineered MSCs and the polyvalent antibody-engineered MSCs under the three shear stress conditions were compared.

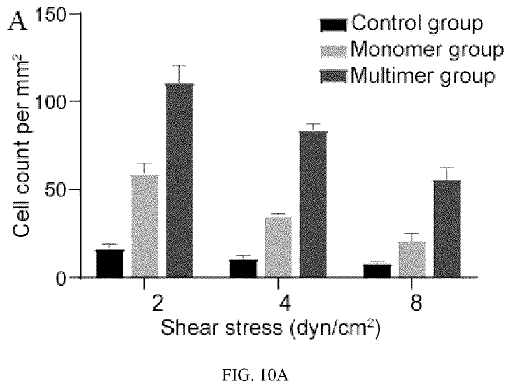

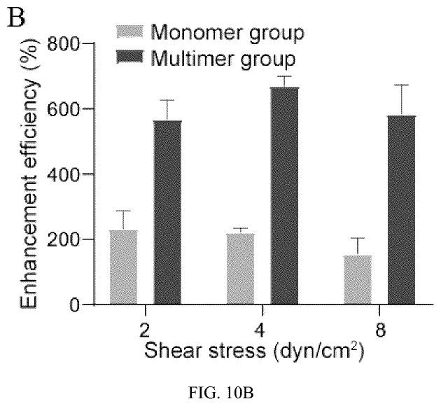

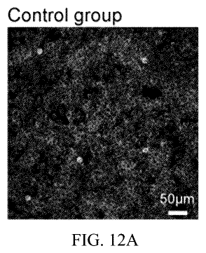

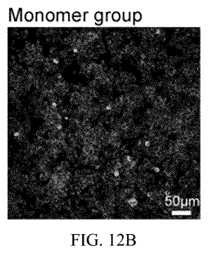

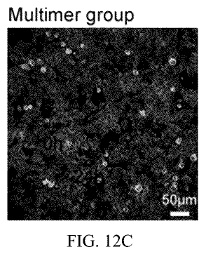

The results showed that under the three shear stress conditions, the number of multimer-engineered MSCs adhered to C166 cells was consistently greater than that of monomer-engineered MSCs, and decreased as the shear stress became greater (FIG. 10A). When the shear stress was 4 dyn/cm², it could be seen that the C166 cells stained with the DID appeared red under fluorescence microscope; and the MSCs stained with the DIO appeared green, indicating that the number of MSCs in the multimer group adhered to C166 cells was significantly higher than that in the control group and the monomer group (FIGS. 12A-12C). When the shear stress was increased from 2 dyn/cm2 to 4 dyn/cm², the adhesion enhancement efficiency of MSCs in the multimer group was also increased in comparison with that in the monomer group, indicating that in the certain range of the shear stress (2 dyn/cm²-8 dyn/cm²), the adhesion effect of multimer-engineered MSCs is better than that of the monomer-engineered MSCs under higher shear stress conditions (FIG. 10B).

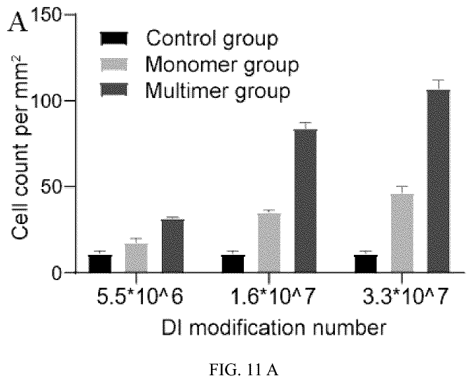

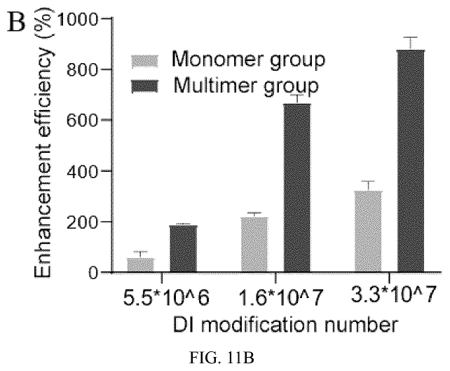

Under the premise of other conditions remaining unchanged, by changing the concentration of DI in step (2) to obtain MSCs engineered with different numbers of DI (5.5×10⁶, 1.6×10⁷, 3.3×10⁷), rolling adhesion assays were performed at a shear stress of 4 dyn/cm² to investigate the effect of numbers of DI on the number of MSCs adhering to the C166 cells. The results showed that when three different numbers of DI molecules were engineered on MSCs, the number of the adhesion cells in the multimer group were than that of the monomer group and the control group; the number of adhesion cells increased with the increase of DI (FIG. 11A); and the adhesion enhancement efficiency of the multimer group was about three times as much as that of the monomer group with three DI number (FIG. 11B).

### Example 8 Migration Test of Engineered MSCs Across Vascular Epithelial Cells

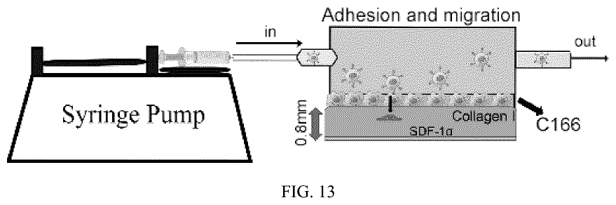

MSCs in vivo would migrate towards the tissues inside the blood vessels after adhering to the blood vessels of damaged tissue site to exert better therapeutic effects. This Example observed whether the MSCs adhered to C166 cells by rolling in Example 7 can migrate downwards cross the C166 cells. The schematic diagram of the experiment device was shown in FIG. 13, where the flow chamber in Example 7 was replaced with a flow chamber for 3D culture, and the rest of the device was the same as in Example 7. Three wells were disposed in the flow chamber. The wells were covered with collagen I. The chemokine SDF-la was added to the collagen I to attract the migration of MSCs. C166 cells were seeded on the collagen I. The MSCs migrated cross the C166 cells, and then migrated to the collagen I for further growth.

The experimental procedure was as follows. 2 μL of stromal cell-derived factor-1 alpha (SDF-1α) solution (purchased from PeproTech, catalog: 250-20A) with a concentration of 500 ng/mL was added to three wells of the flow chamber, followed by adding 15 μL of Collagen I. The wells were incubated at 37° C. for 1 h for the solidification of the collagen. C166 cells with DID staining were seeded in the flow chamber. 5×10⁶ cells were resuspended in 250 μL DMEM complete medium and added to the flow chamber with the syringe. After 4 h, cell adherence was observed under the microscope. 60μL of fresh DMEM complete medium was added to wells of at both sides the flow chamber, and cells were sub-cultured and reached more than 90% coverage after 10 h. The C166 cells could be used to perform the flow adhesion experiment. The 1×10⁶ well-grown MSCs with DIO staining were resuspended in 200 uL of 1×PBS and added with DI (the final concentration was 0.6 μM, and the number of DI fixed on the surface of MSCs was 1.6×10⁷). The cells were mixed well, reacted with oscillating at 25° C. for 20 min, and blown well after 10 min of reaction to obtain a better reaction effect. After the reaction, the cells were centrifuged at 1000 rpm for 5 min, the supernatant was removed, the cells were resuspended in 1 mL of 1×PBS and centrifuged at 1000 rpm for 5 min, the supernatant was removed. The above treatment process was repeated once to wash away the unreacted DI. The MSCs were resuspended in 200 μL of 1×PBS and added with 3 μL of DM1-antiVCAM1 and DM2-antiVCAM1 (prepared in Example 2). The MSCs were mixed well with the pipette gun, reacted with oscillating at 25° C. for 3 h, and blown well every 1 h. After the reaction, the cells were centrifuged at 1000 rpm for 5 min, the supernatant was removed, the cells were resuspended in 1 mL of 1×PBS and centrifuged at 1000 rpm for 5 min, the supernatant was removed. The above treatment process was repeated once to wash away the unreacted antibody, thereby obtaining the engineered MSCs with polyvalent antibody (multimer group). The engineered MSCs obtained by only adding 3 μL of DM1-antiVCAM1 were used as the monomer control group (monomer group, i.e., MSC surface was engineered with antibody monomer). MSCs without any modification were used as the control group. The MSCs in the multimer group, monomer group, and control group were resuspended in 10 mL of 1×PBS and loaded in the 10 ml syringe to perform rolling adhesion assays using the device shown in FIG. 9. The device was connected according to the device schematic diagram. The driving force of the precision syringe pump was adjusted to provide different flow rates, thereby obtaining different shear stresses. The relationship between shear stress and flow rate was calculated according to the following formula: τ=Θ·60.1·Φ, where τ is the shear stress, η is the kinetic viscosity, and Φ is the flow rate. The shear stress condition of 2 dyn/cm² was set. When the flow of 10 mL of cell suspension was finished, the remaining cell suspension in the flow chamber was removed and replaced with DMEM complete medium (0 h). The cells in the flow chamber were imaged in three dimensions using the confocal microscope at 0 h and 24 h, respectively. Further, quantification of MSCs that migrated to collagen I underneath the C166 cells was performed by taking pictures using the confocal microscope. The experiment was repeated three times. 3-5 pictures were taken per time. Image J was used to count the number of MSCs and perform data processing.

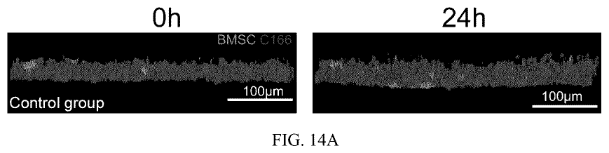

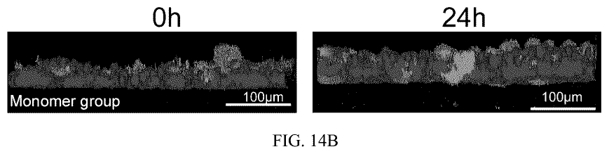

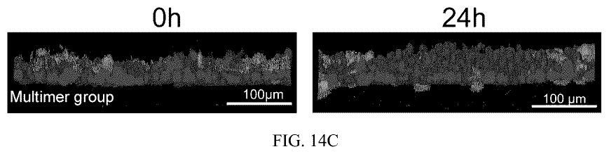

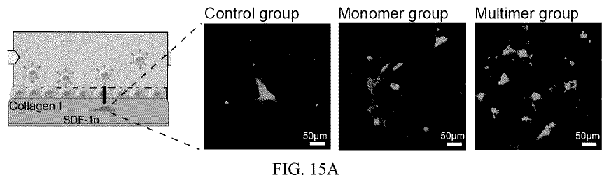

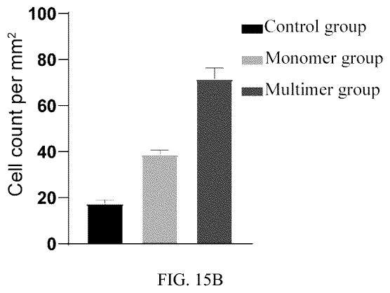

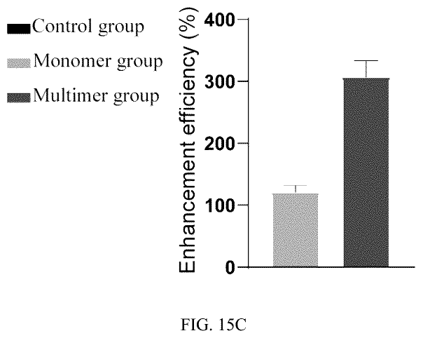

As shown in the 3D imaging results, at 24 h, cells in three groups migrated across the C166 cells (FIGS. 14A-14C). Further, quantification of MSCs migrating to collagen I underneath C166 cells showed that MSCs in the multimer group not only enhanced adhesion to C166 cells, but also enhanced migration to C166 cells. The enhancement efficiency of migration was 2.5 times higher than that of the monomer group (FIGS. 15A-15C), indicating that multimer-engineered MSCs could enhance the adhesion to vascular epithelial cells, thereby enhancing the migration of vascular epithelial cells, and more cells can reach the damaged tissues to exert therapeutic effects.

### Example 9 Proliferation and Paracrine of Engineered MSCs

This Example tested whether MSCs engineered with the polyvalent antibody would influence the proliferation of the cells and their paracrine function.

1. Proliferation of engineered MSCs detected by CCK-8

Well-grown primary mouse bone marrow MSCs were engineered with 1.6×10⁷ and 2.4×10⁷ DI molecules by the method of step (2) of Example 7, respectively. The MSCs with the two DI quantities were then modified with the antibody monomer (DM1-antiVCAM1) or the polyvalent antibody by the method of Example 5 as the experimental groups. The unmodified primary MSCs were used as the control group. A total of 5 groups of MSCs with different treatments were obtained. Each group of treated MSCs was inoculated into 96-well plates at 3000 cells/well respectively, with three replicate wells in each group. Only DMEM low-sugar complete medium without cells was used as a blank control group. Two different 96-well plates were inoculated separately under the same conditions (one was tested after 24 h of incubation, and one was detected after 48 h). After 24 h of incubation, one of the two 96-well plates was taken out, 10μL of CCK8 solution was added to each well and incubated at 37° C. for 1 h. After the reaction was completed, the absorbance value at 450nm (OD) was detected with an enzyme marker. The actual OD values of the experimental group and the control group =OD value detected-OD value of the blank control group. The other 96-well plate was continued to be incubated until 48 h, and the same method was used to detect the OD value of the cells at 450 nm. The OD value of the cells in the control group and the OD value of the experimental group were compared and calculated to obtain the proliferation ratio of the cells in each experimental group.

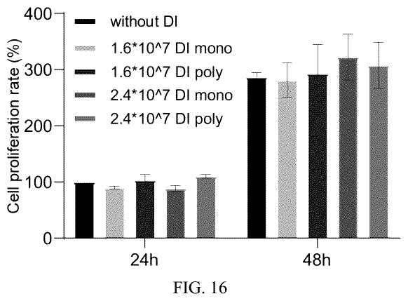

As shown in FIG. 16, the cell proliferation rate of MSCs engineered with either polyvalent antibodies or antibody monomers was not significantly different from that of the control group (MSCs without any modification), indicating that the engineered MSCs did not affect the cell proliferation.

2. Test whether engineered MSCs affect cellular secretion of cytokines by ELISA kit

Eight cytokines secreted by MSCs were selected, such as FGF-2, HGF, SDF-1α, MCP-1, VEGF, TGF-β1, IL-6 and bFGF. Take FGF-2 as an example, the content of cytokines secreted by the engineered MSCs was determined by ELISA kit. The contents of the other cytokines were determined in the same way as that of FGF-2. The experimental steps were as follows.

(1) Well-grown primary MSCs were engineered with 1.6×10⁷ and 2.4×10⁷ DI molecules by the method of Example 6, respectively. The MSCs with the two DI quantities were then modified with the antibody monomer (DM1-antiVCAM1) or the polyvalent antibody by the method of Example 5 as the experimental groups. The primary MSCs without any modification were used as the control group. A total of 5 groups of MSCs with different treatments were obtained. Each group of treated MSCs was inoculated into 12-well plates at 60,000 cells/well respectively, with 3 replicate wells in each group. The cells were placed in an incubator for 4 days, the medium was collected, and the cells were centrifuged at 3,000 rpm and 4° C. for 20 min, and the supernatant was taken and set aside.

(2) The supernatant was assayed using ELISA kits according to the manufacturer's instructions. ELASA kits for FGF-2 were purchased from Shanghai Enzyme-linked Biotechnology Co., Ltd. (catalog: JLC3311). ELISA kits for other cytokines, such as HGF, SDF-1α, MCP-1, VEGF, TGF-β1, IL-6 and bFGF were purchased from Shanghai Enzyme-Link Biotechnology Co., Ltd., and the catalogs of ELISA kits were JLC3897, JLC3170, JLC3907, JLC4130, JLC4255, and JLC6498, respectively.

The manufacturer's instructions were as follows. Dilution of standards: the kit provided one original standard, and the original standard was diluted using standard diluent to the concentration of 1600 ng/L, 800 ng/L, 400 ng/L, 200 ng/L and 100 ng/L, respectively, for standby. Addition of samples: blank wells, standard wells, and sample wells to be tested were set up on the matching coating ELISA plate; no samples and enzyme reagents were added to the blank control wells, and the rest of steps were same; 50 μL of the above standards of different concentrations were added in standard wells;

40 μL of sample diluent was first added, and 10μL of the sample to be tested was then added into sample wells to be tested, that was, the final dilution of the sample to be tested was 5 times; each treatment had 3 replicate wells; and during adding samples, the samples were added to the well bottom of the ELISA plate under gently shaking and mixing, as far as possible not to touch the wall of the wells. Incubation: the ELISA plate was sealed using the sealing film and placed in the incubation of 37° C. for 30 min. Wash: the sealing film was carefully removed, the liquid was removed, the ELISA plate was dried by spin-drying, each well was filled with the washing solution (30 times concentrated washing solution was diluted 30 times with distilled water and then used), the washing solution was left for 30s and removed, wash steps were repeated 5 times, and the ELISA plate was dried. Addition of enzyme: 50 μL of enzyme reagent was added to each well (except for the blank wells). Incubation: the ELISA plate was sealed using the sealing film and placed in the incubation of 37° C. for 30 min. Wash: the sealing film was carefully removed, the liquid was removed, the ELISA plate was dried by spin-drying, each well was filled with the washing solution (30 times concentrated washing solution was diluted 30 times with distilled water and then used), the washing solution was left for 30s and removed, wash steps were repeated 5 times, and the ELISA plate was dried. Color development: 50 μL of color developing reagent A and 50 μL of color developing reagent B were successively added into each well, shook gently and mixed well, and then developed the colors for 10 min at 37° C. away from the light. Termination: 50 μL of terminating solution was added into each well to terminate the reaction (at this time, the blue color would turn to yellow immediately). Measurement: Take the blank well as the zero, the absorbance (OD value) of each well was measured sequentially at 450 nm, and the measurement should be carried out within 15 min after adding the termination solution.

(3) The concentration of the standard was taken as the horizontal coordinate, and the OD value of the standard was taken as the vertical coordinate to plot the standard curve. The OD value of the sample to be tested was substituted into the standard curve to calculate the corresponding concentration. The corresponding concentration was then multiplied by the dilution factor (the dilution factor was 5), thereby obtaining the actual concentration of FGF-2 in the sample to be tested.

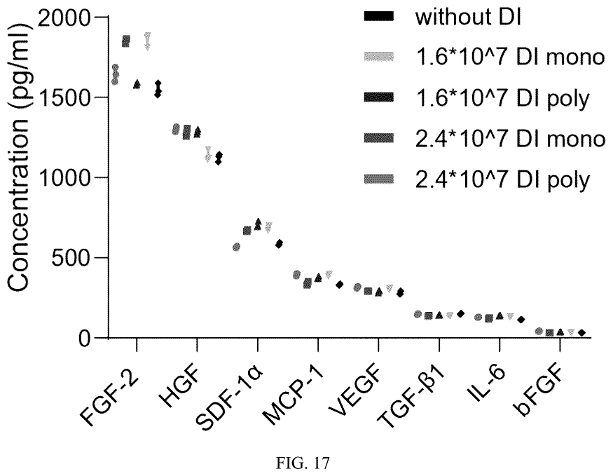

FIG. 17 showed that the number of important cytokines (FGF-2, HGF, SDF-1α, MCP-1, VEGF, TGF-β1, IL-6, and bFGF) secreted by MSCs engineered with either polyvalent antibodies or antibody monomers was not significantly different from that of the control group (MSCs without any modification). This indicated that the engineered MSCs did not affect the paracrine function of the cells.

### Example 10 Adhesion of Engineered MSCs to Damaged Tissues was Observed in vivo

This EXAMPLE was used to detect the adhesion of engineered MSCs to damaged tissue in vivo in mice.

### Construction of Acute Ear Inflammation Model in Mice

Female BALb/c mice of 6-8 weeks and about 20g were selected. 30ug of Escherichia coli-derived lipopolysaccharides (LPS, dissolved in saline up to 1 mg/mL) was injected subcutaneously into the right ear of the mice. An equal volume of saline was injected into the left ear of the mice as a control. The mice were cultured for 6-8 h, and the mouse model with acute ear inflammation was obtained. The corresponding MSCs could be injected at this time.

### Treatment of MSCs

The primary MSCs were performed with DID staining using a staining kit (Beyotime). After staining the MSCs, 1.6×10⁷ DI molecules were engineered on the MSCs according to the method in step (2) of Example 7. Then, the MSCs with DI were then modified by the method of Example 5 with the antibody monomer (DM1-antiVCAM1, monomer group) or the polyvalent antibody (multimer group). The primary MSCs without modification were used as the control group.
1. In-vivo imaging system (IVIS) imaging of mouse ears to observe the cell fluorescence (macroscopic level)

The MSCs from the control group, monomer group and multimer group were injected into mice with acute ear inflammation via tail vein, respectively. Each mouse was injected with 200 μL of cell suspension (containing 1×10⁶ cells). 24 mice were injected in each treatment group. The fluorescence of the mouse ears was observed at 12 h, 24 h, 48 h and 72 h after cell injection. The left and right ears of the mice were imaged in vivo at the corresponding time points using the IVIS Small Animal In Vivo Imager, respectively.

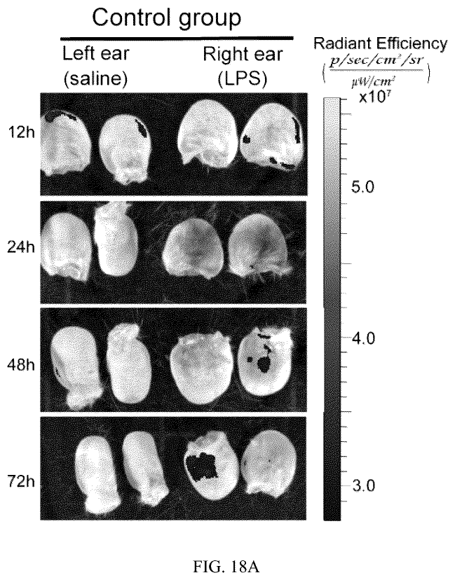

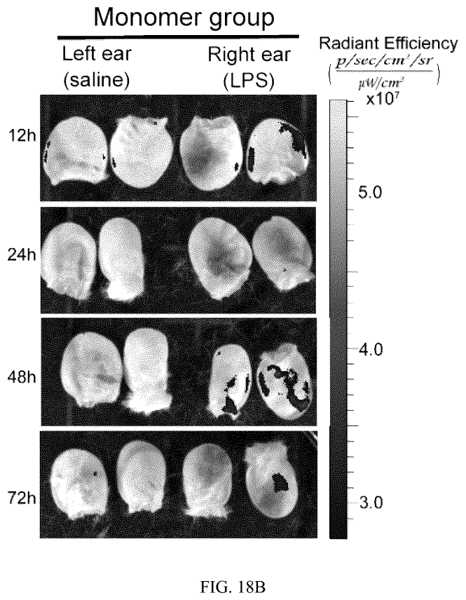

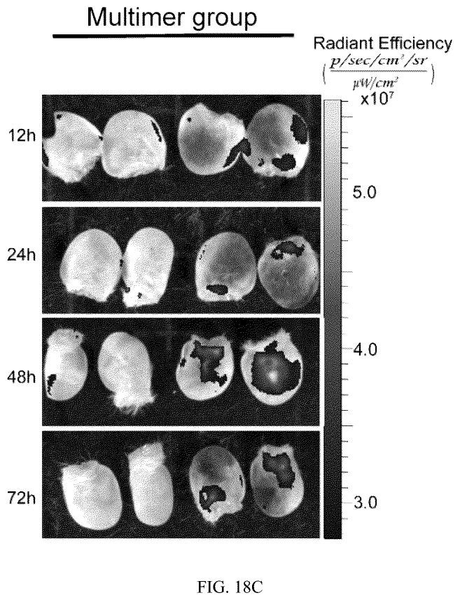

FIGS. 18A-18C showed the number of MSCs adhering to the mouse ears in the multimer group was higher than that in the monomer group and the control group at the four time points of 12 h, 24 h, 48 h and 72 h. The strongest fluorescence appeared at 48 h, indicating that the most MSCs homed to the mouse ears at that time point. The difference of the multimer group from the monomer group and the control group was also the most obvious at that time point (48 h), indicating that the polyvalent antibody-engineered MSCs had a higher number of cells homing to damaged tissues in mice than either antibody-monomer-engineered MSCs or MSCs without any modifications.
2. Confocal microscopy observation of MSCs adhering in the blood vessels of mouse ears (microscopic level)

The MSCs from the control group, monomer group and multimer group were injected into mice with acute ear inflammation via tail vein, respectively. Each mouse was injected with 200 μL of cell suspension (containing 1×10⁶ cells). After 48 h, 100 μL of 10 mg/mL FITC-dextran solution (Hangzhou Xinqiao Bio-technology Co., Ltd, molecular weight 2000 of kDa) was injected into mice in each group via tail vein. After 2 h, the mice were euthanized. The right ears of the mice were taken for confocal fluorescence microscopy imaging.

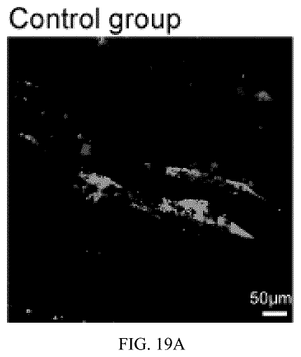

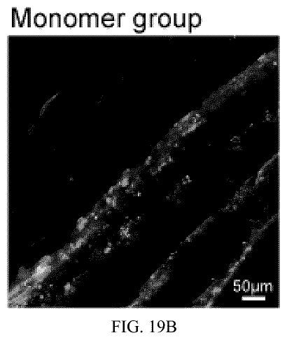

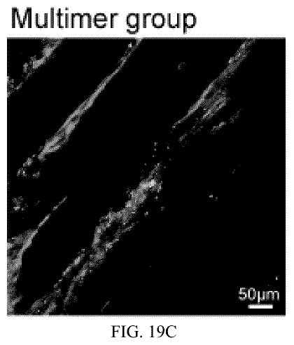

In FIGS. 19A-19C, the green represented mouse blood vessels labelled by FITC-dextran, and the red represented MSCs. FIGS. 19A-19C showed that the number of MSCs adhering to the blood vessels in the multimer group was significantly higher than that in the monomer group and the control group. Moreover, more MSCs in the multimer group homed to the inflammation site because more cells adhered to the blood vessels. These results indicated that the polyvalent antibody-engineered MSCs enhanced the ability of the cells to home to the inflammation site by enhancing the adhesion to the vascular epithelial cells, which enhanced the therapeutic effect of the cells.
3. Flow assay for the number of MSCs in mouse ears in each group

The MSCs from the control group, monomer group and multimer group were injected into mice with acute ear inflammation via tail vein, respectively. Each mouse was injected with 200 μL of cell suspension (containing 1×10⁶ cells). After 48 h, the mice were euthanized, the mouse ears were disassembled into single cells through the following steps. The mouse ears were clipped along the base, and the hair was removed. The mouse ears were cut to the smallest size and broken down into single cells in a 1.5 mL Eppendorf (EP) tube. 1 mL of tissue digestion solution was added into the single cells, and the single cells were reacted with oscillating at 37° C. for 1 h. The tissue digestion solution contained 1640 medium containing 0.1 mg/mL DNase I, 0.2 mg/mL Liberase TL (Sigma Aldrich, catalog: L3024) and 1 v/v % FBS. At the end of the reaction, the undigested ear tissue was inverted and fully ground in a 70 um cell strainer. During the grinding process, the cell strainer was rinsed with an appropriate amount of tissue rinse solution (1×PBS containing 2 mM EDTA and 1v/v % FBS) until no large pieces of tissue could be seen in the grinding process. Then, finally the tissue rinse solution was added for rinse. The digested tissue liquid and the liquid obtained after the grinding and rinsing process were combined and centrifuged at 1000 rpm for 5 min, and the supernatant was removed. The single cells were taken at the lower layer, washed once with 1×PBS (pH 7.4), and centrifuged again, and the supernatant was removed. The single cells were transferred to a 1.5 mL EP tube, and 1 mL of erythrocyte lysate was added. The lysate was incubated on ice for 2 min, and centrifuged at 1,000 rpm for 5 min, and the supernatant was removed, washed once with PBS, and centrifuged again for supernatant removal. The cells were resuspended in 0.5 mL 1×PBS containing DAPI (5 mg/mL of DAPI mother liquor was mixed with 1×PBS according to volume ratio 1:6000) and detected using flow cytometer.

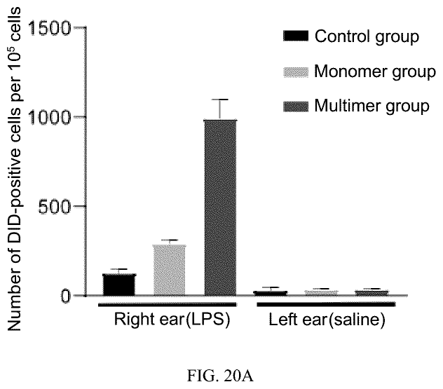

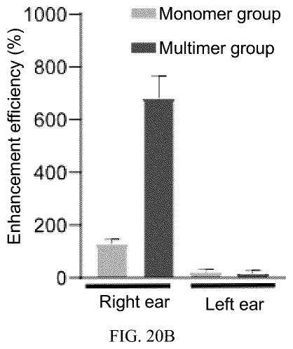

FIGS. 20A-20B showed that the number of MSCs adhering to the inflammation site in the multimer group was greater than that of the cells in the monomer group and the control group, which was about 3.5 times greater than that in the monomer group and 7.8 times greater than that in the control group. The enhancement efficiency of adhesion in the multimer group (calculated by the same formula as in Example 7) was about 5 times higher than that in the monomer group. Quantification of MSCs in ears of the mice with inflammation further illustrated the enhanced homing ability of polyvalent antibody-engineered MSCs to inflammation sites.
4. Section observation of engineered MSCs adhesion to the blood vessels of damaged tissues

The MSCs from the control group, monomer group and multimer group were injected into mice with acute ear inflammation via tail vein, respectively. Each mouse was injected with 200 μL of cell suspension (containing 1×10⁶ cells). After 48 h, the mice were euthanized, and the ears of mice were removed, and the ears of the mice with inflammation were sectioned using the frozen section method. The sections were stained with the vascular-labelled antibody FITC anti-CD31 (Biolengend, catalog: 102506). The specific steps were as follows. Fixation: the mouse ears were cut down along the base, and the hairs were removed. The tissue was fixed in 4% paraformaldehyde and taken out after 5 h. The paraformaldehyde was washed away with 1×PBS to avoid contamination of subsequent experimental manipulations. The tissue was placed in 30% sucrose and dehydrated overnight. Embedding: a plastic mold was placed for embedding on dry ice. A layer of Optimal Cutting Temperature embedding compound (OCT) was first spread on the mold. The tissue was laid flat onto the OCT. The tissue was covered with OCT glue and placed on the dry ice until the OCT was solidified. The embedded tissue was placed in dry ice and taken to a frozen slicer to cut into tissue sections with a thickness of 8 um. The tissue sections were placed on polylysine-treated slides with slightly press so that the tissue sections were adsorbed flatly to the center of the slide and quickly placed in dry ice for storage. Staining: the slides were placed on a shaker and washed 3-5 times with 1×PBS for 5 min every time to remove the OCT. The slides were placed in a wet box and sealed with a sealing solution (Beyotime, catalog: P0252) for 1 h at room temperature. The sealing solution was removed. The water around the tissue was dried using the clean absorbent paper. The slides were placed in the wet box and added dropwise with diluted FITC anti-CD31 antibody (diluted 1:100 with primary antibody diluent (Bioengineering (Shanghai) Co., Ltd., catalog: E674004)) for overnight at 4° C. Sealing: after the overnight reaction, the antibody on the slides was washed off with 1×PBS and washed with 1×PBS on a shaking table for 3-5 times, each time for 5 min. The water on the tissues was absorbed with the clean absorbent paper. The sealing agent (Anti-fluorescence quenching polyvinylpyrrolidone (PVP) sealing solution, Beyotime, catalog: P0123) was added dropwise on the tissues and gently sealed the sections with a coverslip, avoiding bubbles. The sealed tissue sections were placed under a fluorescence microscope for observation.

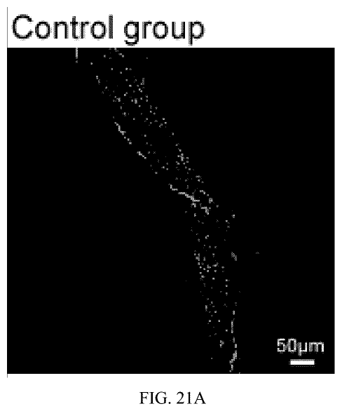

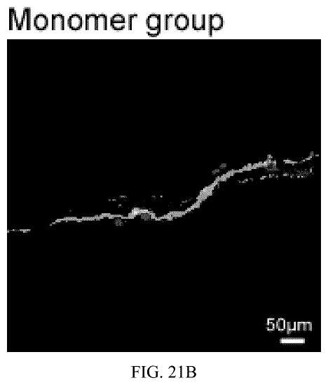

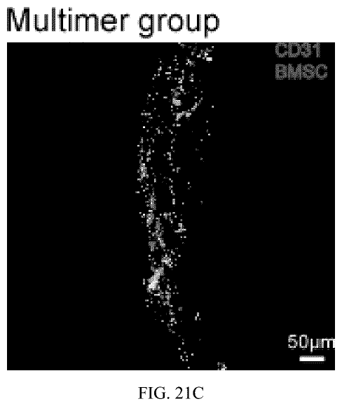

Sections of the ears of mice with inflammation were cut by the frozen section method. The sections were stained with the antibody FITC anti-CD31 that labelled blood vessels. The adhesion of the engineered MSCs on the blood vessels of the ears could be observed in the fluorescence microscope due to DID staining of the engineered MSCs prior to injection. Red color indicated MSCs, and green color indicated the antibody FITC anti-CD31. FIGS. 21A-21C showed that the number of MSCs in the multimer group adhered to the blood vessels of mice was more than that in the monomer group and the control group.

The animal experiments indicated that the intravenous injection of MSCs into the body after being engineered by polyvalent antibodies can significantly increase the adhesion of MSCs to vascular endothelial cells, thus improving the homing ability of the cells to the damaged site. After applying the polyvalent antibody to the engineered modification of MSCs, the adhesion of MSCs to vascular endothelial cells can be greatly enhanced, the homing ability of MSCs can be improved, and a better tissue repair function can be exerted.

The embodiments of the present disclosure have been described in detail above in conjunction with the accompanying drawings, not intended to limit the disclosure. Various changes can be made by those skilled in the art without departing from the spirit of the disclosure. The embodiments of the present disclosure and the features in the embodiments may be combined with each other without conflict.

What is claimed is:

1. An antibody monomer, comprising:
an antibody; and
a first DNA monomer or a second DNA monomer;
wherein a nucleotide sequence of the first DNA monomer consists of SEQ ID NO: 1; a nucleotide sequence of the second DNA monomer consists of SEQ ID NO: 2; and the antibody is an anti-vascular cell adhesion molecule 1 (antiVCAM1) antibody.

2. A kit, comprising:
the antibody monomer of claim 1.

3. The kit of claim 2, further comprising:
an initiator.

4. The kit of claim 3, wherein the initiator comprises a nucleotide sequence consisting of SEQ ID NO:3.

5. The kit of claim 3, wherein a cholesterol moiety is linked to 3′ end of the nucleotide sequence of the initiator.

6. A polyvalent antibody, comprising:
a first antibody monomer; and
a second antibody monomer;
wherein the first antibody monomer comprises a first DNA monomer and a first antibody; the second antibody monomer comprises a second DNA monomer and a second antibody; a nucleotide sequence of the first DNA monomer consists of SEQ ID NO: 1; and a nucleotide sequence of the second DNA monomer consists of SEQ ID NO: 2; and the first antibody and the second antibody are each an anti-vascular cell adhesion molecule 1 (antiVCAM1) antibody; and
the first antibody monomer and the second antibody monomer are connected through a hybridization chain reaction.

7. A cell, wherein the cell is a mesenchymal stem cell surface-modified with the antibody monomer of claim 1.

8. A cell, wherein the cell is a mesenchymal stem cell surface-modified with the polyvalent antibody of claim 6.

9. A cell engineering method, comprising:
(a) mixing a to-be-engineered cell with an initiator followed by a first reaction to obtain a reaction product; and
(b) mixing the reaction product with the antibody monomer of claim 1 followed by a second reaction to obtain an engineered cell; and
wherein the to-be-engineered cell is a mesenchymal stem cell, and the initiator comprises a nucleotide sequence consisting of SEQ ID NO: 3.

10. The method of claim 9, wherein a final concentration of the initiator in a mixture of the to-be-engineered cell and the initiator is 0.05-3.00 μM.

11. The method of claim 9, wherein the first reaction is performed under shaking at 20-37° C. for 15-30 min.

12. The method of claim 9, wherein the second reaction is performed under shaking at 20-37° C. for 2-4 h.

13. A cell engineering method, comprising:
(1) mixing a to-be-engineered cell with an initiator followed by a first reaction to obtain a reaction product; and
(2) mixing the reaction product with the polyvalent antibody of claim 6 followed by a second reaction to obtain an engineered cell; and
wherein the to-be-engineered cell is a mesenchymal stem cell, and the initiator comprises a nucleotide sequence consisting of SEQ ID NO: 3.

14. The method of claim 13, wherein a final concentration of the initiator in a mixture of the to-be-engineered cell and the initiator is 0.05-3.00 μM.

15. The method of claim 13, wherein the first reaction is performed under shaking at 20-37° C. for 15-30 min.

16. The method of claim 13, wherein the second reaction is performed under shaking at 20-37° C. for 2-4 h.

17. A method of treating damaged tissue in a subject in need thereof, comprising:
administering the cell of claim 7 to the subject.

18. A method of treating damaged tissue in a subject in need thereof, comprising:
administering the cell of claim 8 to the subject.


SEQUENCE LISTING (3 sequences)

SEQ ID NO: 1 - DNA, 48 bp, synthetic construct
  source 1..48: mol_type = other DNA
ggtttaggtaggagtgggatgaggccaaatcctcatcccactcctacc

SEQ ID NO: 2 - DNA, 48 bp, synthetic construct
  source 1..48: mol_type = other DNA
cctcatcccactcctacctaaaccggtaggagtgggatgaggatttgg

SEQ ID NO: 3 - DNA, 44 bp, synthetic construct
  source 1..44: mol_type = other DNA
cctcatcccactcctacctaaacctttttttttttttttttttt